(19) United States
(12) Patent Application Publication
(10) Pub. No.: US 2022/0065791 A1
(43) Pub. Date: Mar. 3, 2022

(54) STIMULATED RAMAN SPECTROSCOPY BASED MULTIPLEXED VIRTUAL IMMUNOHISTOLOGY USING ALKYNIC, NITRILE, OR AZIDE PROBES

(71) Applicant: CytoVeris Inc., Farmington, CT (US)

(72) Inventors: Alan Kersey, South Glastonbury, CT (US); Rishikesh Pandey, Unionville, CT (US)

(21) Appl. No.: 17/464,380

(22) Filed: Sep. 1, 2021

Related U.S. Application Data
(60) Provisional application No. 63/072,964, filed on Sep. 1, 2020.

(51) Int. Cl. G01N 21/65; G01J 3/44; G01N 33/483

(52) U.S. Cl. CPC G01N 21/65; G01N 2021/655; G01N 33/4833; G01J 3/4412

(57) ABSTRACT

A system for and method of examining a tissue sample using stimulated Raman spectroscopy is provided. The method includes: a) producing a first beam of light at a first wavelength; b) producing a second beam of light at at least a second wavelength, the second wavelength different from the first wavelength; c) combining the first and second beams of light to provide a combined output; d) interrogating a tissue sample with the combined output to produce Raman scattering light, the tissue sample prepared with at least one target molecule having a targeting agent conjugated with a Raman silent dye, the targeting agent configured to bind with at least one biomarker; e) detecting at least a portion of the produced Raman scattering light using a photodetector; and f) producing immunohistological data relating to the tissue sample using photodetector signals representative of the detected Raman scattering light.

This application claims priority to U.S. Patent Appln. No. 63/072,964 filed Sep. 1, 2020, which is hereby incorporated by reference in its entirety.

### BACKGROUND OF THE INVENTION

### 1. Technical Field

The present disclosure relates to systems and methods for examining a tissue sample using Raman spectroscopy in general, and more specifically to systems and methods for examining a tissue sample using stimulated Raman spectroscopy

### 2. Background Information

For many decades the reference method for the diagnosis of cancer has been histopathological examination of tissues using conventional microscopy. This process is known as Surgical Pathology. In Surgical Pathology, samples can be produced from surgical procedures (tumor resection), diagnostic biopsies or autopsies. These samples go through a process that includes dissection, fixation, and cutting of tissue into precisely thin slices which are stained for contrast and mounted onto glass slides. The slides are subsequently examined by a pathologist under a microscope, and their interpretations of the tissue results in the pathology “read” of the sample. Advanced optical and electromagnetic (“EM”) imaging approaches have been reported for the determination of tumor margin. These approaches include the use of fluorescence imaging [1-2], near-infrared spectroscopy [3], terahertz reflectivity [4], Raman spectroscopy [5-7], and the like.

Raman spectroscopy measures the inelastic scattering of light and provides molecular-specific information. A Raman spectrum shows the intensity of scattered light with different wavelength shifts, usually denoted in wavenumbers (cm⁻¹). Owing to probe water-containing samples with rich molecular-specific information content coupled with no or minimal sample preparation requirement, Raman spectroscopy has now emerged as a powerful tool for biomedical applications [8]. Raman spectroscopy has been employed to investigate biospecimens such as live cells, tissues [9], body fluids, etc. Clinical applications include in vivo biopsy guidance [10], cancer detection in an intraoperative setting [6], blood analyte monitoring [11], and the like.

While Raman spectroscopy provides molecular fingerprinting information, only one in about one hundred million (1 in ˜10⁸) photons are inelastically scattered. This results in a lower signal-to-noise ratio or necessitates a long signal acquisition time. Several approaches have been employed to enhance intrinsically weak Raman signals and thereby increase the detection sensitivity of Raman spectroscopy. These approaches include resonance Raman [12], Surface-enhanced Raman scattering (SERS) [13], coherent anti-Stokes Raman scattering (CARS) [14], and stimulated Raman scattering (SRS) [15].

SRS is a technique in which the sample is coherently excited with two lasers namely a pump laser (ω_{P}) and a Stokes frequency laser (ω_{S}). The stimulated transition occurs when the frequency difference between the output of the aforesaid lasers (ω_{P}−ω_{S}) matches a Raman-active molecular vibration. Traditionally, SRS has been utilized to probe a single Raman band but investigating multiple Raman peaks has become feasible and practical. SRS has been successfully used to acquire high-contrast molecular-specific images with a very high acquisition speed even with video rate [16]. SRS has been proposed for the intraoperative uses [17]. While SRS has been proposed for tissue imaging, it has been primarily been restricted to high wavenumber region and some cases in the fingerprint region.

SRS has been primarily used as a label-free tool by only exploiting the intrinsic molecular contrast and chemical bonds such as C—H, O—H, C═O, C═C, etc. However, detection specificity has been limited due to presence of the same chemical bond in other biomolecules resulting in overlapping signal. SRS with exogenous Raman-tag have been proposed to increase the targeted and specific probing [18].

The quantification of protein cancer biomarkers of cancer has been proposed to detect tumors. To this end, multiple biomarkers have been proposed to identify breast cancer with high sensitivity [19]. A surface-enhanced based approach in the fingerprint region has been proposed for intraoperative lumpectomy surgical guidance by simultaneously measuring a large panel of protein biomarkers [20].

Several moieties such as alkynes, nitriles, and azides feature relatively narrow Raman signatures in the Raman-silent region (1800-2800 cm−1) [21]. FIG. 1 illustrates Raman spectra of three major breast tissue types (adipose, benign, and cancerous) showing different spectral regions; i.e., the fingerprint region (“FP Region”), the silent region, and high-wave number (“HWN” region). Consequently, Raman reporters with these moieties allow imaging of tissues and cells with no or negligible interference from the biomolecular constituents. However, as can be seen in FIG. 1 the region between 1800-2800 cm⁻¹ is nearly featureless in all the three Raman spectra.

### SUMMARY

According to an aspect of the present disclosure, a method of examining a tissue sample using stimulated Raman spectroscopy is provided. The method includes: a) producing a first beam of light at a first wavelength using a pump laser; b) producing a second beam of light at at least a second wavelength, the second wavelength different from the first wavelength; c) combining the first beam of light and the second beam of light to provide a combined output; d) interrogating a tissue sample with the combined output to produce Raman scattering light, the tissue sample prepared with at least one target molecule having a targeting agent conjugated with a Raman silent dye (RSD), the targeting agent configured to bind with at least one biomarker; e) detecting at least a portion of the produced Raman scattering light using at least one photodetector, the photodetector producing signals representative of the detected Raman scattering light; and f) producing immunohistological data relating to the tissue sample using the signals representative of the detected Raman scattering light.

In any of the aspects or embodiments described above and herein, the step of producing immunohistological data may include determining a presence of at least one said biomarker.

In any of the aspects or embodiments described above and herein, the step of producing immunohistological data may include quantifying at least one biomarker determined to be present.

In any of the aspects or embodiments described above and herein, the at least one biomarker may be an indicator of a presence of cancerous tissue.

In any of the aspects or embodiments described above and herein, the tissue sample may be prepared with a plurality of different target molecules, wherein the targeting agent of each target molecule is different from the targeting agent of the other targeting molecules, and each respective targeting agent is conjugated with a different RSD, wherein each RSD produces Raman scattering light, and each RSD produces Raman scattering light that is distinguishable from the Raman scattering light produced by the other RSDs.

In any of the aspects or embodiments described above and herein, each RSD may produce Raman scattering light in the Raman silent region, and each RSD may produce Raman scattering light in the Raman silent region that is distinguishable from the Raman scattering light in the Raman silent region produced by the other RSDs.

In any of the aspects or embodiments described above and herein, further comprising a step of filtering the produced Raman scattering light using a plurality of narrow band filters, each respective narrow band filter configured to pass a portion of the produced Raman scattering light associated with a wavenumber in the Raman silent region, and the portion passed by each respective narrow band filter is different from the portion passed by the other of the narrow band filters and is associated with a different wavenumber in the Raman silent region.

In any of the aspects or embodiments described above and herein, further comprising a step of filtering the produced Raman scattering light using a controllable narrow band filter, wherein the controllable narrow band filter is sequentially operated to pass a plurality of different portions of the produced Raman scattering light, each respective portion associated with a different wavenumber in the Raman silent region.

In any of the aspects or embodiments described above and herein, wherein the step of detecting may utilize one photodetector, the photodetector producing signals representative of the sequentially detected Raman scattering light.

In any of the aspects or embodiments described above and herein, wherein the step of producing a second beam of light may utilize a light source that produces a continuum of light containing light at “N” different wavelengths, where “N” is an integer equal to two or more, and the “N” different wavelengths includes the second wavelength.

In any of the aspects or embodiments described above and herein, wherein the step of producing a second beam of light further may include controlling a light source to sequentially produce the second beam of light at “N” different wavelengths, where “N” is an integer equal to two or more, and the “N” different wavelengths includes the second wavelength.

In any of the aspects or embodiments described above and herein, wherein the step of producing a second beam of light may include controlling a plurality of different light sources, each respective light source configured to produce a beam of light at wavelength different from the other plurality of different light sources, to sequentially produce the second beam of light at “N” different wavelengths, where “N” is an integer equal to two or more, and the “N” different wavelengths includes the second wavelength.

In any of the aspects or embodiments described above and herein, the tissue sample may be an ex vivo tissue sample.

According to an aspect of the present disclosure, a system for examining a tissue sample using stimulated Raman spectroscopy is provided that includes a pump laser, a Stokes beam source, a plurality of optical elements, at least one photodetector, and a control unit. The pump laser is configured to produce a first beam of light at a first wavelength. The Stokes beam source is configured to produce a second beam of light at at least a second wavelength, the second wavelength different from the first wavelength. The at least one photodetector is configured to detect Raman scattering light and produce signals representative of the detected Raman scattering light. The control unit is in communication with pump laser, the Stokes beam source, the at least one photodetector, the plurality of optical elements, and a non-transitory memory storing instructions. The instructions when executed cause the processor to: a) control the pump laser, the Stokes beam source, and at least one of the plurality of optical elements to produce a combined output using the first beam of light and the second beam of light; b) cause a tissue sample prepared with at least one target molecule having a targeting agent conjugated with a Raman silent dye (RSD), the targeting agent configured to bind with at least one biomarker, to be interrogated with the combined output and produce Raman scattering light as a result of the interrogation; c) control the at least one photodetector to detect at least a portion of the Raman scattering light and produce signals representative of the detected Raman scattering light; and d) produce immunohistological data relating to the tissue sample using the signals representative of the detected Raman scattering light.

In any of the aspects or embodiments described above and herein, the instructions that cause the processor to determine the immunohistological data may further cause the processor to determine a presence of at least one said biomarker.

In any of the aspects or embodiments described above and herein, the instructions that cause the processor to determine the immunohistological data may further cause the processor to quantify a biomarker determined to be present.

In any of the aspects or embodiments described above and herein, a biomarker may be an indicator of a presence of cancerous tissue.

In any of the aspects or embodiments described above and herein, the tissue sample may be prepared with a plurality of different target molecules, wherein the targeting agent of each target molecule is different from the targeting agent of the other targeting molecules, and each respective targeting agent is conjugated with a different RSD, wherein each RSD produces Raman scattering light, and each RSD produces Raman scattering light that is distinguishable from the Raman scattering light produced by the other RSDs.

In any of the aspects or embodiments described above and herein, each RSD may produce Raman scattering light in the Raman silent region, and each RSD may produce Raman scattering light in the Raman silent region that is distinguishable from the Raman scattering light in the Raman silent region produced by the other RSDs.

In any of the aspects or embodiments described above and herein, the system may further include a plurality of narrow band filters configured to filter the produced Raman scattering light, wherein each respective narrow band filter is configured to pass a portion of the produced Raman scattering light associated with a wavenumber in the Raman silent region, and the portion passed by each respective narrow band filter is different from the portion passed by the other of the narrow band filters and is associated with a different wavenumber in the Raman silent region.

In any of the aspects or embodiments described above and herein, the system may further include a controllable narrow band filter configured to filter the produced Raman scattering light, and the instructions when executed may cause the processor to control the controllable narrow band filter to sequentially pass a plurality of different portions of the produced Raman scattering light, each respective portion associated with a different wavenumber in the Raman silent region.

In any of the aspects or embodiments described above and herein, wherein the Stokes beam source may be configured to produce a continuum of light containing light at “N” different wavelengths, where “N” is an integer equal to two or more, and the “N” different wavelengths includes the second wavelength.

In any of the aspects or embodiments described above and herein, wherein the Stokes beam source may be controllable to sequentially produce the second beam of light at “N” different wavelengths, where “N” is an integer equal to two or more, and the “N” different wavelengths includes the second wavelength.

In any of the aspects or embodiments described above and herein, wherein the Stokes beam source may include a plurality of different light sources, each respective light source configured to produce a beam of light at wavelength different from the other plurality of different light sources, to sequentially produce the second beam of light at “N” different wavelengths, where “N” is an integer equal to two or more, and the “N” different wavelengths includes the second wavelength.

According to another aspect of the present disclosure, a method of examining a tissue sample using stimulated Raman spectroscopy is provided. The method includes: a) preparing a tissue sample with at least one target molecule having a targeting agent conjugated with a Raman silent dye (RSD), the targeting agent configured to bind with at least one biomarker; b) producing a first beam of light at a first wavelength using a pump laser; c) producing a second beam of light at at least a second wavelength, the second wavelength different from the first wavelength; d) combining the first beam of light and the second beam of light to provide a combined output; e) interrogating the prepared tissue sample with the combined output to produce Raman scattering light; f) detecting at least a portion of the produced Raman scattering light using at least one photodetector, the photodetector producing signals representative of the detected Raman scattering light; and g) producing immunohistological data relating to the tissue sample using the signals representative of the detected Raman scattering light.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a graph of normalized intensity versus wavenumber that illustrates Raman spectra of three major breast tissue types (adipose, benign, and cancerous) showing different spectral regions; i.e., the fingerprint region (“FP Region”), the silent region, and high-wave number (“HWN” region).

FIG. 2 is a schematic illustration of present disclosure embodiment steps.

FIG. 3 is an example of a present disclosure system embodiment.

FIG. 4 is an example of a present disclosure system embodiment.

FIG. 5 is an example of a present disclosure system embodiment.

### DETAILED DESCRIPTION

Aspects of the present disclosure include a novel and non-obvious system 20 and method for using stimulated Raman scattering (SRS) to examine a tissue sample for the presence or absence of cancerous tissue.

The present disclosure utilizes molecules (referred to hereinafter as “target molecules 22”) that target biomarkers of interest. A non-limiting biomarker of interest is one that is typically overexpressed by cancerous tissue. To facilitate the description herein, the biomarkers are referred to herein as “cancer biomarkers” as the present disclosure provides significant benefits in the cancer detection field. The present disclosure is not, however, limited to biomarkers indicative of cancerous tissue. The term “cancer biomarker” as used herein refers to a biological substance (e.g., a protein, etc.) that the presence, or the increased concentration, of is an indicator of the presence of cancerous tissue. The biological substance may or may not be present within the tissue normally. If present within the tissue, the concentration of the biological substance may normally at a very low level even to the point where the biological substance is undetectable using conventional techniques. These biological substances may be “overexpressed” by cancerous tissue, resulting in a high concentration of the biological substance (relative to normal) and that relatively high concentration provides an indication of the presence of cancerous tissue. Hence, they may be used as “cancer biomarkers”. The specific type of cancer biomarker(s) may vary depending upon the type of cancer, but the specific types of cancer biomarkers associated with specific types of cancer are known. Non-limiting examples of breast cancer biomarkers include estrogen receptor (ER), human epidermal growth factor receptor 2 (HER2), epidermal growth factor receptor (EGFR), and CD44. The present disclosure may be configured to target a variety of different cancer biomarkers and is not therefore limited to any particular cancer biomarker.

Aspects of the present disclosure include the production of and/or use of target molecules 22 that include a targeting agent conjugated with a Raman silent dye (RSD). A targeting agent may be any molecule or construct such as an antibody, an affibody, or the like configured to target a cancer biomarker of interest. The targeting agent may be described as a vehicle for selectively delivering an RSD to cancerous tissue. In some applications, a plurality of different target molecules 22 (e.g., a first target molecule 22 having a first targeting agent, a second target molecule 22 having a second targeting agent, etc.) may be used to permit a multiplexed detection of cancer biomarkers. The target molecules 22 provide desirable cancer detection specificity and permit quantification of cancer biomarker expression. Non-limiting examples of targeting agents include antibodies, affibodies, and the like. With regard to those embodiments of the present disclosure that may be used to detect breast cancer, examples of targeting agents include antibodies, affibodies, etc.

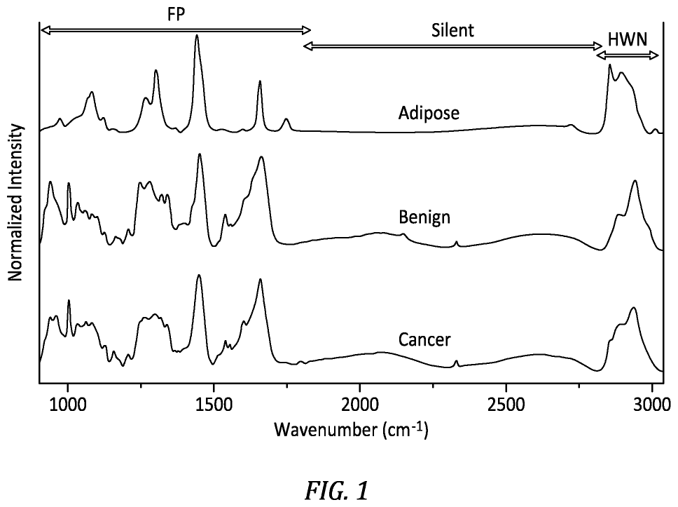

RSDs are configured to produce the Raman spectrum in the Raman-silent region when subjected to the one or more predetermined wavelengths of light. Raman spectra of endogenous biospecies are typically negligible in the silent region as shown in FIG. 1. Hence, the RSD provides a spectrum that is distinct in the Raman-silent region and facilitates identification. The RSD may include an alkyne, a nitrile, or an azide moiety, or the like conjugated with a targeting agent.

As will be evident from the exemplary system 20 embodiments disclosed herein, the present disclosure target molecules 22 can provide a simplified means of cancerous tissue detection. For example, embodiments of the present disclosure may utilize one or more filters that are configured, or can be operated, to pass only certain Raman spectral peak signal(s) in the Raman silent region. Each of these Raman spectral peaks may be associated with a particular RSD. This facile filter-based detection approach facilitates an elegant Raman “imaging” system that may use a photodetector without the need for a spectrometer or other spectral analysis device.

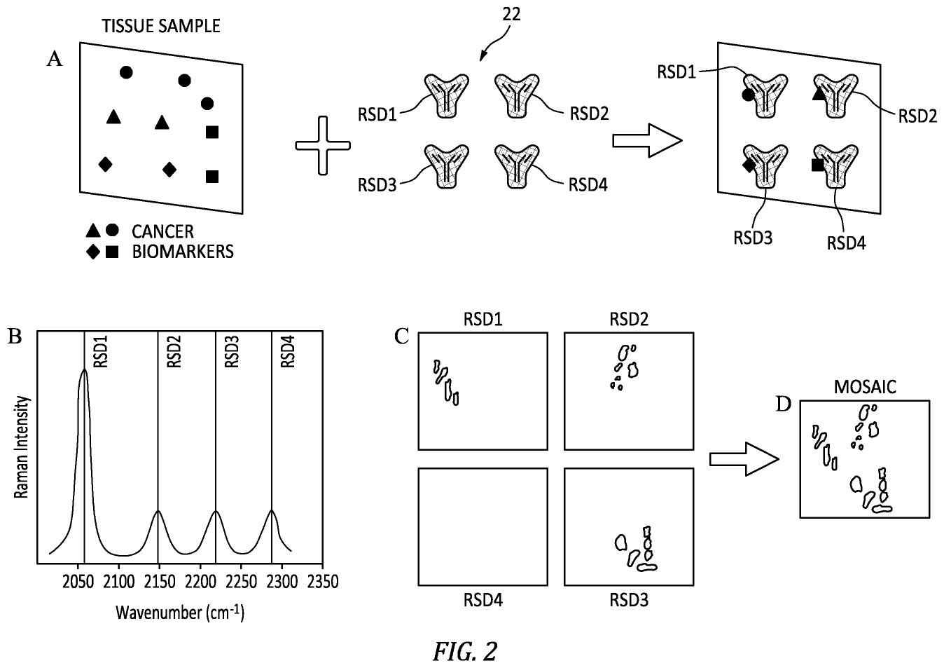

A schematic illustration of embodiments of the present disclosure is shown in FIG. 2. A solution containing a plurality of target molecules 22 with different RSDs (i.e., RSD1, RSD2, RSD3, RSD4) is applied (e.g., topically) to a tissue sample. The present disclosure may be used for ex vivo or in vivo tissue samples. To facilitate the description herein, the tissue will be described hereinafter as an ex vivo tissue sample. After a period of time sufficient for the target molecules 22 present within the solution to bind to any targeted cancer biomarkers that may be present, the tissue sample may be “washed” using a buffer solution such as phosphate-buffered saline (PBS) or the like to remove any unbound target molecules 22. The aforesaid wash solution may in some embodiments include other constituents to facilitate the examination; e.g., bovine serum albumin to reduce non-specific binding. After the unbound target molecules 22 are removed, the tissue sample may be examined in a manner described below. FIG. 2 schematically illustrates the use of a plurality of different RSDs (i.e., RSD1, RSD2, RSD3, RSD4) which permits multi-color imaging. The present disclosure is not limited to RSDs that produce different colors in multi-color imaging; i.e., other imaging techniques that permit the RSDs to be distinguishable from one another may be used. To facilitate the description herein, the RSDs will be described as producing different colors when imaged. In the “A” portion of the black and white FIG. 2, the different colors of the RSDs are depicted by different cross-hatching. The multicolor RSDs provide immunohistochemical information due to the selective detection of a cancer biomarker specific to the conjugated targeting agent (e.g., antibody). The multiplexed detection may be realized by simultaneous recording of “N” different moiety peaks (e.g., from different alkynes, or azides, or nitriles, or any combination thereof, etc.) in the Raman silent region; e.g., each alkyne peak associated with a different wavenumber in the Raman silent region as shown in portion “B” of FIG. 2. As will be apparent from the description below, the aforesaid peaks may be captured using a single photodetector that can be operated to capture a plurality of select alkyne (or the like) peaks, or “N” individual photodetectors (where “N” is an integer). In the latter configuration having “N” photodetectors, each photodetector may be coupled with a unique filter configured to record only a chosen alkyne (or the like) peak. Importantly, using such a strategy permits simultaneous and multiplexed recording of the non-overlapping Raman signals from the alkynes/azides/nitriles with high specificity and the detection of more than one cancer biomarker. The “C” portion of FIG. 2 depicts four squares, each representing an SRS image associated with a different target molecule/RSD (i.e., RSD1, RSD2, RSD3, RSD4) and therefore with a different cancer biomarker. The SRS image depiction labeled “RSD1” illustrates some evidence of the presence of the cancer biomarker targeted by the target molecule including RSD1. The SRS image depiction labeled “RSD2” illustrates some evidence of the presence of the cancer biomarker targeted by the target molecule including RSD2. The SRS image depiction labeled “RSD3” illustrates some evidence of the presence of the cancer biomarker targeted by the target molecule including RSD3. The SRS image depiction labeled “RSD4” illustrates no evidence of the presence of the cancer biomarker targeted by the target molecule including RSD4. The SRS image depiction labeled “Mosaic” depicts the cancer biomarker presence information from RSD1-RSD4 as a combined depiction, or “mosaic”.

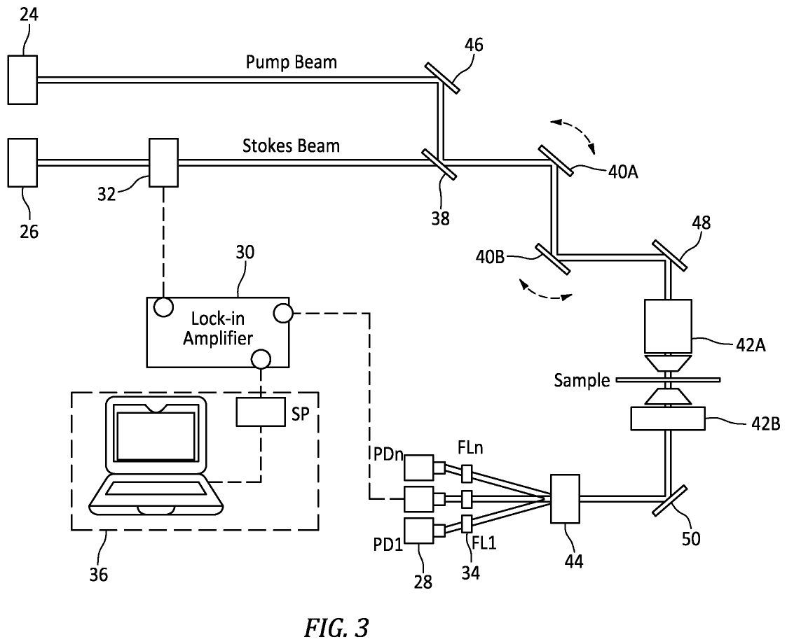

Exemplary embodiments of the present disclosure system 20 are shown in FIGS. 3-5. These system 20 embodiments include a pump laser 24, a Stokes beam source 26, at least one photodetector 28, a lock-in amplifier 30, an electro-optical modulator or an acousto-optic modulator (collectively referred to hereinafter as “EOM/AOM 32”), at least one narrow band filter 34, various mirrors, and a control unit 36. In some embodiments, the lock-in amplifier 30, the “EOM/AOM 32”), the at least one narrow band filter 34, and the mirrors may be referred to as optical elements. Pump lasers, which produce a non-continuous output, are well known. The pump laser 24 produces a light beam output (ω_{P}) typically at a single predetermined wavelength. The Stokes beam source 26 (sometimes referred to as a “probe”) may be configured to produce a beam of light having a continuum of wavelengths (e.g., a bandwidth containing a plurality of wavelengths), or to produce a beam of light at a single wavelength, or it may be a device that can be tuned to provide a plurality of beams of light each at a different wavelength, or it may include a plurality of light sources (e.g., lasers) each producing a beam of light at a different wavelength, or the like. The system 20 embodiments shown in FIGS. 3-5 and described below utilize examples of different Stokes beam source 26 embodiments. As will be described below, the present disclosure system 20 embodiments are configured to operate in a stimulated Raman scattering (SRS) mode wherein the pump laser output (ω_{P}) is combined with the Stokes beam output (ω_{S}). The pump laser output (ω_{P}) and the Stokes beam output (ω_{S}) are chosen to produce a combined output (cop-cos) that is used as an excitation light to interrogate the tissue sample. The properties of the pump laser output (ω_{P}) and the Stokes beam output (ω_{S}) are chosen so that the combined output (cop-cos) coincides with a Raman-active molecular vibration of a constituent element of the targeted cancer biomarker. A change in the Stokes beam output (ω_{S}) results in a change in the combined output (cop-cos), thereby permitting more than one Raman-active molecular vibration to be targeted; i.e., more than one cancer biomarker. The lock-in amplifier 30 and EOM/AOM 32 function to modulate the Stokes beam (ω_{S}) and the modulation may be imposed on the phase, frequency, amplitude, or polarization of the Stokes beam. The lock-in detection enables measuring the change in pump beam (stimulated Raman loss) due to SRS process at the detector. In different embodiments not shown here, the pump beam can also be modulated and the Stokes signal may be changed to provide stimulated Raman information (stimulated Raman gain). In the diagrammatic system 20 embodiments shown in FIGS. 3-5, the lock-in amplifier 30 is shown independent of the control unit 36. In alternative embodiments, the lock-in amplifier 30 may be incorporated into the control unit 36 or the functionality of the lock-in amplifier 30 may be accomplished by the control unit 36.

The at least one photodetector 28 is configured to receive Raman scattered light produced as a result of the interrogation of the tissue sample by the combined output excitation light. The photodetector 28 can be chosen to provide optimal performance at the wavelength(s) of light passed by the filter, and at the typically low intensity of the light. Non-limiting examples of an acceptable photodetector 28 include avalanche photodiodes, CCD arrays, and the like. In some embodiments, the light intensity captured at each photodetector 28 may be integrated for a time duration “T” to increase the effective signal to noise ratio.

The filter(s), various mirrors, and other optical components will be detailed in the embodiments shown in FIGS. 3-5.

The control unit 36 in communication with other components within the system 20, such as the pump laser 24, the Stokes beam source 26, the photodetector(s) 28, the lock-in amplifier 30, and the electro-optical modulator, and may be in communication with the filter(s) and mirrors, to control the functions of the respective components; e.g., communicate signals to and/or from the respective components to perform the functions described herein. The control unit 36 (and other components within the system 20) may include any type of computing device, computational circuit, processor(s), CPU, GPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system 20 to accomplish the same algorithmically and/or coordination of system components. The control unit 36 may include a single memory device or a plurality of memory devices. The present disclosure is not limited to any particular type of non-transitory memory device, and may include read-only memory, random access memory, volatile memory, non-volatile memory, static memory, dynamic memory, flash memory, cache memory, and/or any device that stores digital information. The control unit 36 may include one or more interfaces that permit communication with an input device that enables a user to enter data and/or instructions, and/or an output device configured, for example to display information (e.g., a visual display or a printer), or to transfer data, etc. In some embodiments, input and/or output devices may be incorporated into the control unit 36. Communications between the control unit 36 and other system 20 components may be via a hardwire connection or via a wireless connection.

The system 20 embodiment diagrammatically shown in FIG. 3 includes a pump laser 24, a Stokes beam source 26, a plurality of photodetectors 28, a lock-in amplifier 30, an EOM/AOM 32, a plurality of narrow band filters 34, mirrors, a dichroic mirror 38, a pair of galvo mirrors 40A, 40B, an objective lens 42A, 42B, a beam splitter 44, and a control unit 36. The Stokes beam source 26 is configured to produce a beam of light having a continuum of wavelengths. The EOM/AOM 32 and the lock-in amplifier 30 are configured (by themselves or in communication with the control unit 36) to coordinate the Stokes beam output (cos; typically, in the range of about 2000 to about 2400 cm⁻¹) and the pump laser output (ω_{P}) to permit production of the combined output (ω_{P}−ω_{S}). The system 20 is configured so that the beam of light produced by the pump laser 24 (“pump laser beam”) is incident to a first mirror 46 which directs the pump laser beam to strike (and reflect off of) a dichroic mirror 38. The system 20 is further configured so that the beam of light produced by the Stokes beam source 26 (“Stokes beam”) is incident to the dichroic mirror 38 and passes through the dichroic mirror 38 coincident with the pump laser beam reflected by the dichroic mirror 38. Hence, the pump laser beam and the Stokes beam are combined upon exiting the dichroic mirror 38 to form the combined output (ω_{P}−ω_{S}). The combined output is incident to the first galvo mirror 40A which in turn reflects the combined output to be incident to a second galvo mirror 40B. The second galvo mirror 40B reflects the combined output to be incident to a second mirror 48 which in turn reflects the combined output to a first objective lens 42A. The tissue sample is disposed such that combined output passing through the first objective lens 42A is incident to the tissue sample.

The first and second galvo mirrors 40A, 40B are in communication with the control unit 36. The control unit 36 may control the galvo mirrors 40A, 40B so that the combined output incident to the tissue sample is positionally movable relative to the tissue sample; e.g., the galvo mirrors 40A, 40B can be controlled to cause the combined output to scan at least a portion of the tissue sample. The present disclosure is not limited to using galvo mirrors to scan the combined output relative to the tissue sample. For example, in some embodiments the system 20 may omit the galvo mirrors in favor of a movable stage that is controllable to move the tissue sample relative to the combined output; e.g., at least a portion of the tissue sample may be scanned by moving the stage.

As described above, the tissue sample has been prepared with a solution containing some number of target molecules 22, each including a targeting agent conjugated with a Raman silent dye (RSD). For example, the solution may include three different types of target molecules 22, each with a distinct targeting agent (for binding with a specific cancer biomarker) and a distinct alkyne that produces a different photometric response in the Raman silent region. The preparation of the tissue sample includes washing unbound target molecules 22 from the tissue sample after an acceptable binding period of time, thereby leaving only those target molecules 22 (if any) that are bound to cancer biomarkers present in the tissue sample (if any). The bound target molecules 22 indicate the presence of cancerous tissue and can be used to identify the location of the same on the tissue sample.

Raman scattering light produced by the combined output interrogation of the tissue sample is collected by the second objective lens 42B and passed to a third mirror 50. The third mirror 50 in turn reflects the collected Raman scattering to a beam splitter 44. The beam splitter 44 splits the collected Raman scattering into “N” portions, where “N” is an integer. In the example shown in FIG. 3, the beam splitter 44 is shown splitting the collected Raman scattering into “N” (shown as three) portions. A respective narrow band filter 34 is disposed to receive a respective portion of the Raman scattering. Each narrow band filter 34 is configured to block light (e.g., Raman scattering, all excitation light, etc.) other than the Raman scattering associated with a particular predetermined wavenumber peak in the Raman silent region (e.g., one of “N” alkyne peaks if the target molecules 22 include “N” different alkynes). In this manner, the present disclosure system 20 permits multiplexed detection of “N” wavenumber peaks (and therefore “N” cancer biomarkers) by simultaneous recording of “N” different alkyne (or azide, or nitriles, etc.) nonoverlapping peaks in the Raman silent region along “N” separate channels; e.g., each alkyne peak associated with a different wavenumber in the Raman silent region. The output of each respective narrow band filter 34 is captured by a photodetector 28. Each photodetector 28, in turn, produces signals representative of the captured Raman scattering and communicates those signals to control unit 36. The control unit 36 is configured to analyze the photodetector 28 signals to produce the related wavenumber peak data in the silent region. The aforesaid wavenumber peak data may be used subsequently to produce information regarding the presence or absence of the cancer biomarkers with the tissue sample, and/or pathology information or the like.

The system 20 embodiment shown in FIG. 3 permits peak detection in the Raman silent region by utilizing a Stokes beam source 26 chosen that produces a continuum consisting of frequencies in 2000-2400 cm⁻¹ range with respect to the pump laser 24. In this example, for a 785 nm pump laser, the Stokes beam source 26 may produce light in the frequency range of 931-967 nm. It should be noted that the use of other filters may be used (e.g., as shown) to limit the optical interferences of other scattered light and direct Raman light to, or to block the excitation light from, the detection path.

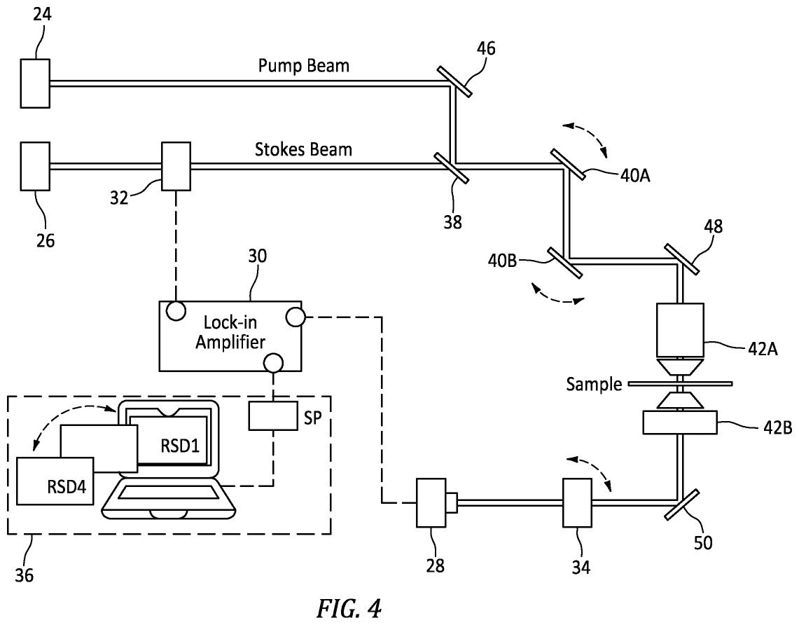

Another system 20 embodiment example is diagrammatically shown in FIG. 4. This system 20 embodiment utilizes a form of SRS detection in a sequential fashion. As will be described below, a narrow band filter 34 that is configured to rotate may be used to allow access to detect differing wavenumber bands of the Raman signature to be detected in a sequential fashion.

The system 20 embodiment diagrammatically shown in FIG. 4 includes a pump laser 24, a Stokes beam source 26, a single photodetector 28, a lock-in amplifier 30, an EOM/AOM 32, a single narrow band filter 34, mirrors, a dichroic mirror 38, a pair of galvo mirrors 40A, 40B, an objective lens 42A, 42B, and a control unit 36. The Stokes beam source 26, the EOM/AOM 32, lock-in amplifier 30, and pump laser 24 are configured in a manner similar to or the same as described above with respect to the system 20 shown in FIG. 3. This system 20 embodiment is configured so that the beam of light produced by the pump laser 24 (“pump laser beam”) is incident to a first mirror 46 which directs the pump laser beam to strike (and reflect off of) a dichroic mirror 38. The system 20 is further configured so that the beam of light produced by the Stokes beam source 26 (“Stokes beam”) is incident to the dichroic mirror 38 and passes through the dichroic mirror 38 coincident with the pump laser beam reflected by the dichroic mirror 38. Hence, the pump laser beam and the Stokes beam are combined upon exiting the dichroic mirror 38 to form the combined output (ω_{P}−ω_{S}). The combined output is directed by the first and second galvo mirrors 40A, 40B in a manner similar to or the same as described above in the system 20 embodiment shown in FIG. 3. The tissue sample is disposed and prepared in a manner similar to or the same as described above in the system 20 embodiment shown in FIG. 3.

Raman scattering light produced by the combined output interrogation of the tissue sample is collected by the second objective lens 42B and passed to a third mirror 50. The third mirror 50 in turn reflects the collected Raman scattering to narrow band filter 34 that is controlled (e.g., rotated) to selectively allow portions of the collected Raman scattering associated with different wavenumber bands (e.g., “N” wavenumber bands) to pass through. In this manner, selective detection of individual wavenumber peaks is permitted in a sequential fashion. Each portion of collected scattering light passed through the narrow band filter 34 continues and is detected by the photodetector 28. The photodetector 28, in turn, produces signals representative of the captured Raman scattering and communicates those signals to control unit 36. The control unit 36 is configured to analyze the photodetector 28 signals to produce the related wavenumber peak data in the silent region. The aforesaid wavenumber peak data may be used subsequently to produce information regarding the presence or absence of the cancer biomarkers with the tissue sample, and/or pathology information or the like.

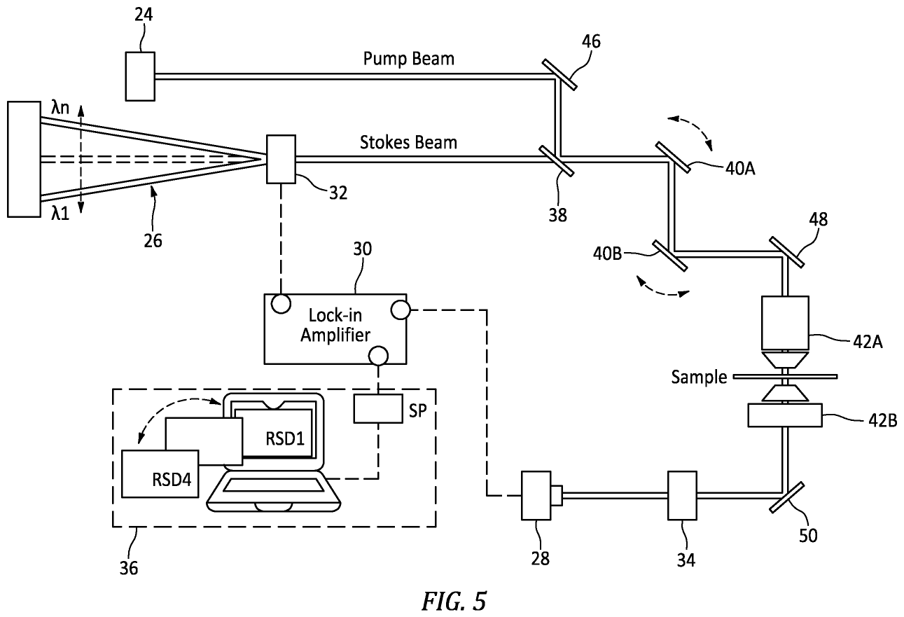

Another system 20 embodiment example is diagrammatically shown in FIG. 5. This system 20 embodiment utilizes a form of SRS detection in a sequential fashion using multiple lasers, each emitting at different frequencies as a Stokes beam source 26 and a single photodetector 28 and narrow band filter 34.

The system 20 embodiment diagrammatically shown in FIG. 5 includes a pump laser 24, a Stokes beam source 26 in the form of a tunable light source or a plurality of different lasers, each configured to emit light at a different wavelength, a single photodetector 28, a lock-in amplifier 30, an EOM/AOM 32, a single narrow band filter 34, mirrors, a dichroic mirror 38, a pair of galvo mirrors 40A, 40B, an objective lens 42A, 42B, and a control unit 36.

The Stokes beam source 26 in this system 20 embodiment is controlled to sequentially produce light at different wavelengths. As stated above, this embodiment may utilize a tunable light source that can be controlled to produce a beam of light at a “N” number of wavelengths, each different from the other. Alternatively, the Stokes beam source 26 may include “N” number of independent lasers, each configured to produce light at a wavelength different from the other, and all controllable to be operated sequentially. The wavelengths of the respective lasers are chosen based on known characteristics of the RSDs. The EOM/AOM 32, lock-in amplifier 30, and pump laser 24 are configured in a manner similar to or the same as described above with respect to the system 20 shown in FIG. 3. This system 20 embodiment is configured to produce the combined output of the pump laser beam and the Stokes beam and direct the combined output through the objective lens 42A, 42B in the manner described above with respect to the system 20 shown in FIG. 3. The tissue sample is disposed and prepared in a manner similar to or the same as described above in the system 20 embodiment shown in FIG. 3. This system 20 embodiment is configured to filter the combined output using a controllable narrow band filter 34 and collect the Raman scattered light using a single photodetector 28 as described above with respect to the system 20 shown in FIG. 4. The control unit 36 is configured to analyze the photodetector 28 signals to produce the related wavenumber peak data in the silent region. The aforesaid wavenumber peak data may be used subsequently to produce information regarding the presence or absence of the cancer biomarkers with the tissue sample, and/or pathology information or the like.

The detailed description of various embodiments herein refers to the accompanying drawings, which show various embodiments by way of illustration. While these various embodiments are described in sufficient detail to enable those skilled in the art to practice the inventions, it should be understood that other embodiments may be realized and that logical, chemical, and mechanical changes may be made without departing from the spirit and scope of the inventions. Thus, the detailed description herein is presented for purposes of illustration only and not of limitation. For example, the steps recited in any of the method or process descriptions may be executed in any order and are not necessarily limited to the order presented.

Furthermore, it is noted that various method or process steps for embodiments of the present disclosure are described in the following description and drawings. The description may present the method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

### REFERENCES

1. Nguyen and Stein, “Fluorescence-guided surgery with live molecular navigation—a new cutting edge”, Nat Rev Cancer, 13(9), pp. 653-662, 2013.
2. Timers, et al., “Real-time intraoperative detection of breast cancer using near-infrared fluorescence imaging and methylene blue”, Eur J Surg Oncol., 40(7), 850-858, 2014.
3. Dhār et al., “A diffuse reflectance spectral imaging system for tumor margin assessment using custom annular photodiode arrays”, Biomedical Optics Express, 3, (12), 2012.
4. Yaroslaysky. A, et al., “Delineating nonmelanoma skin cancer margins using terahertz and optical imaging”, J of Biomedical Photonics & Eng., 3(1), 2017.
5. Pence I., Mahadevan-Jansen A., “Clinical instrumentation and applications of Raman spectroscopy”, Chem Soc Rev.; 45 (7):1958-1979, 2016.
6. Talari, A. et al., “Raman Spectroscopy of Biological Tissues”, Applied Spectroscopy Reviews, 50:1, 46-111, 2015.
7. A. S. Haka, et al., “In vivo margin assessment during partial mastectomy breast surgery using Raman spectroscopy,” Cancer Res. 66(6), 3317-3322 (2006).
8. S. Anwar et al., “Advanced Raman spectroscopy from research to clinic” Translational potential and challenges”, Spectrochimica Acta Part A: Molecular and Biomolecular Spectroscopy, Vol. 260, 5 Nov. 2021 (available online 13 May 2021). https://www.sciencedirect.com/science/article/pii/S1386142521005345
9. U.S. Patent Publication No. 2017/0020460, “System and Method for Assessing a Cancer Status of Biological Tissue”, Leblond et al.
10. J. Desroches et al., “Development and first in-human use of a Raman spectroscopy guidance system integrated with a brain biopsy needle”, J Biophotonics, 2019 March; https://pubmed.ncbi.nlm.nih.gov/30636032
11. R. Pandey et al., “Label-free spectrochemical probe for determination of hemoglobin glycation in clinical blood samples”, J Biophotonics, 3 May 2018; https://onlinelibrary.wiley.com/doi/abs/10.1002/jbio.201700397
12. Y. Zhou, et al., “Optical biopsy identification and grading of gliomas using label-free visible resonance Raman spectroscopy,” J. of Biomedical Optics, 24(9), 095001 (2019).
13. Y. Wang, et al., “Raman-Encoded Molecular Imaging with Topically Applied SERS Nanoparticles for Intraoperative Guidance of Lumpectomy,” Cancer Res. 77(16), 4506-4516(2017).
14. C. Zhang et al., “Coherent Raman Scattering Microscopy in Biology and Medicine”, Annu Rev Biomed Eng. 2015, Dec. 7; 17: 415-445; https://www.ncbi.nlm.nih.gov/pmc/articles/PMC4773899
15. T. C. Hollon et al., “Near real-time intraoperative brain tumor diagnosis using stimulated Raman histology and deep neural networks”, Nature Medicine, 26, 52-58(2020)
16. Z. Zhao et al., “Applications of vibrational tags in biological imaging by Raman spectroscopy”, Analyst. 2017 Oct. 23; 142(21): 4018-4029; https://www.ncbi.nlm.nih.gov/pmc/articles/PMC5674523
17. M. Ji et al., “Rapid, label-free detection of brain tumors with stimulated Raman scattering microscopy”, Sci Transl Med. 2013 Sep. 4; 5(210): 201ra119; https://www.ncbi.nlm.nih.gov/pmc/articles/PMC3806096/18.
18. L. Wei et al., “Live-cell imaging of alkyne-tagged small biomolecules by stimulated Raman scattering”, Nature Methods, 11, 410-412 (2014); https://www.nature.com/articles/nmeth.2878?proof=t
19. M. V. Yezhelyev et al., “In Situ Molecular Profiling of Breast Cancer Biomarkers with Multicolor Quantum Dots”, Advanced Materials, 3146-3151; 17 Oct. 2007; https://onlinelibrary.wiley.com/doi/abs/10.1002/adma.200701983
20. Y. W. Wang et al., “Raman-Encoded Molecular Imaging with Topically Applied SERS Nanoparticles for Intraoperative Guidance of Lumpectomy”, Cancer Res. 2017 Aug. 15; 77(16); 4506-4516; https://pubmed.ncbi.nlm.nih.gov/28615226
21. L. Wei et al., “Live-cell imaging of alkyne-tagged small biomolecules by stimulated Raman scattering”, Nature Methods, 11, 410-412(2014

1. A method of examining a tissue sample using stimulated Raman spectroscopy, comprising:
producing a first beam of light at a first wavelength using a pump laser;
producing a second beam of light at at least a second wavelength, the second wavelength different from the first wavelength;
combining the first beam of light and the second beam of light to provide a combined output;
interrogating a tissue sample with the combined output to produce Raman scattering light, the tissue sample prepared with at least one target molecule having a targeting agent conjugated with a Raman silent dye (RSD), the targeting agent configured to bind with at least one biomarker;
detecting at least a portion of the produced Raman scattering light using at least one photodetector, the photodetector producing signals representative of the detected Raman scattering light; and
producing immunohistological data relating to the tissue sample using the signals representative of the detected Raman scattering light.

2. The method of claim 1, wherein the step of producing immunohistological data includes determining a presence of at least one said biomarker.

3. The method of claim 2, wherein the step of producing immunohistological data includes quantifying said at least one said at least one biomarker determined to be present.

4. The method of claim 1, wherein the at least one biomarker is an indicator of a presence of cancerous tissue.

5. The method of claim 1, wherein the tissue sample is prepared with a plurality of different said target molecules, wherein the targeting agent of each said target molecule is different from the targeting agent of the other said targeting molecules, and each respective targeting agent is conjugated with a different said RSD, wherein each said RSD produces Raman scattering light, and each RSD produces Raman scattering light that is distinguishable from the Raman scattering light produced by the other RSDs.

6. The method of claim 5, wherein each said RSD produces Raman scattering light in the Raman silent region, and each RSD produces Raman scattering light in the Raman silent region that is distinguishable from the Raman scattering light in the Raman silent region produced by the other RSDs.

7. The method of claim 5, further comprising a step of filtering the produced Raman scattering light using a plurality of narrow band filters, each respective narrow band filter of the plurality of narrow band filters configured to pass a portion of the produced Raman scattering light associated with a wavenumber in the Raman silent region, and the portion passed by each respective narrow band filter is different from the portion passed by the other of the narrow band filters and is associated with a different said wavenumber in the Raman silent region.

8. The method of claim 5, further comprising a step of filtering the produced Raman scattering light using a controllable narrow band filter, wherein the controllable narrow band filter is sequentially operated to pass a plurality of different portions of the produced Raman scattering light, each respective portion associated with a different wavenumber in the Raman silent region.

9. The method of claim 8, wherein the step of detecting utilizes one photodetector, the photodetector producing signals representative of the sequentially detected Raman scattering light.

10. The method of claim 1, wherein the step of producing a second beam of light utilizes a light source that produces a continuum of light containing light at “N” different wavelengths, where “N” is an integer equal to two or more, and the “N” different wavelengths includes the second wavelength.

11. The method of claim 1, wherein the step of producing a second beam of light further includes controlling a light source to sequentially produce the second beam of light at “N” different wavelengths, where “N” is an integer equal to two or more, and the “N” different wavelengths includes the second wavelength.

12. The method of claim 1, wherein the step of producing a second beam of light includes controlling a plurality of different light sources, each respective said light source configured to produce a beam of light at wavelength different from the other said plurality of different light sources, to sequentially produce the second beam of light at “N” different wavelengths, where “N” is an integer equal to two or more, and the “N” different wavelengths includes the second wavelength.

13. The method of claim 1, wherein the tissue sample is an ex vivo tissue sample.

14. A system for examining a tissue sample using stimulated Raman spectroscopy, comprising:
a pump laser configured to produce a first beam of light at a first wavelength;
a Stokes beam source configured to produce a second beam of light at at least a second wavelength, the second wavelength different from the first wavelength;
a plurality of optical elements;
at least one photodetector configured to detect Raman scattering light and produce signals representative of the detected Raman scattering light; and
a control unit in communication with pump laser, the Stokes beam source, the at least one photodetector, the plurality of optical elements, and a non-transitory memory storing instructions, which instructions when executed cause the processor to:
control the pump laser, the Stokes beam source, and at least one of the plurality of optical elements to produce a combined output using the first beam of light and the second beam of light;
cause a tissue sample prepared with at least one target molecule having a targeting agent conjugated with a Raman silent dye (RSD), the targeting agent configured to bind with at least one biomarker, to be interrogated with the combined output and produce Raman scattering light as a result of the interrogation;
control the at least one photodetector to detect at least a portion of the Raman scattering light and produce signals representative of the detected Raman scattering light; and
produce immunohistological data relating to the tissue sample using the signals representative of the detected Raman scattering light.

15. The system of claim 14, wherein the instructions that cause the processor to determine said immunohistological data further cause the processor to determine a presence of at least one said biomarker.

16. The system of claim 15, wherein the instructions that cause the processor to determine said immunohistological data further cause the processor to quantify said at least one said at least one biomarker determined to be present.

17. The system of claim 14, wherein the at least one biomarker is an indicator of a presence of cancerous tissue.

18. The system of claim 14, wherein the tissue sample is prepared with a plurality of different said target molecules, wherein the targeting agent of each said target molecule is different from the targeting agent of the other said targeting molecules, and each respective targeting agent is conjugated with a different said RSD, wherein each said RSD produces Raman scattering light, and each RSD produces Raman scattering light that is distinguishable from the Raman scattering light produced by the other RSDs.

19. The system of claim 18, wherein each said RSD produces Raman scattering light in the Raman silent region, and each RSD produces Raman scattering light in the Raman silent region that is distinguishable from the Raman scattering light in the Raman silent region produced by the other RSDs.

20. The system of claim 18, further comprising a plurality of narrow band filters configured to filter the produced Raman scattering light, wherein each respective narrow band filter of the plurality of narrow band filters is configured to pass a portion of the produced Raman scattering light associated with a wavenumber in the Raman silent region, and the portion passed by each respective narrow band filter is different from the portion passed by the other of the narrow band filters and is associated with a different said wavenumber in the Raman silent region.

21. The system of claim 18, further comprising a controllable narrow band filter configured to filter the produced Raman scattering light; and
wherein the instructions when executed cause the processor to control the controllable narrow band filter to sequentially pass a plurality of different portions of the produced Raman scattering light, each respective portion associated with a different wavenumber in the Raman silent region.

22. The system of claim 14, wherein the Stokes beam source is configured to produce a continuum of light containing light at “N” different wavelengths, where “N” is an integer equal to two or more, and the “N” different wavelengths includes the second wavelength.

23. The system of claim 14, wherein the Stokes beam source is controllable to sequentially produce the second beam of light at “N” different wavelengths, where “N” is an integer equal to two or more, and the “N” different wavelengths includes the second wavelength.

24. The system of claim 14, wherein the Stokes beam source includes a plurality of different light sources, each respective said light source configured to produce a beam of light at wavelength different from the other said plurality of different light sources, to sequentially produce the second beam of light at “N” different wavelengths, where “N” is an integer equal to two or more, and the “N” different wavelengths includes the second wavelength.

25. A method of examining a tissue sample using stimulated Raman spectroscopy, comprising:
preparing a tissue sample with at least one target molecule having a targeting agent conjugated with a Raman silent dye (RSD), the targeting agent configured to bind with at least one biomarker;
producing a first beam of light at a first wavelength using a pump laser;
producing a second beam of light at at least a second wavelength, the second wavelength different from the first wavelength;
combining the first beam of light and the second beam of light to provide a combined output;
interrogating the prepared tissue sample with the combined output to produce Raman scattering light;
detecting at least a portion of the produced Raman scattering light using at least one photodetector, the photodetector producing signals representative of the detected Raman scattering light; and
producing immunohistological data relating to the tissue sample using the signals representative of the detected Raman scattering light.
